(19) United States
(12) Patent Application Publication
(10) Pub. No.: US 2026/0076576 A1
(43) Pub. Date: Mar. 19, 2026

(54) SYSTEM AND METHOD TO CALCULATE PHYSIOLOGICAL PARAMETERS FROM HUMAN BODY USING MULTIPLE COMMUNICATION DEVICE SENSORS SIMULTANEOUSLY

(71) Applicant: Jashna Oza, San Jose, CA (US)

(72) Inventor: Jashna Oza, San Jose, CA (US)

(21) Appl. No.: 18/885,665

(22) Filed: Sep. 15, 2024

(51) Int. Cl. A61B 5/00

(52) U.S. Cl. CPC A61B 5/0077; A61B 5/0002; A61B 5/0064; A61B 5/0082; A61B 5/6898; A61B 5/7203; A61B 2560/0462; A61B 2562/0204

(57) ABSTRACT

The embodiments herein provide a method and a system for calculating physiological parameters from human body using multiple communication device sensors simultaneously. The method for calculating physiological parameters from human body using multiple communication device sensors simultaneously includes collecting inputs from one or more of a plurality of cameras, microphones and sensors in a communication device for enhancing the accuracy of the measurement of the physiological parameters. The method further includes, using at least one of the camera, microphone and sensor of the communication device for measuring the physiological parameters by placing one or more fingertips on rear and/or front cameras and microphone or using the front camera to capture facial video data and audio data from fingertips. The method further includes, measuring a plurality of physiological parameters simultaneously or asynchronously using a combination of audio video plethysmography, signal processing and machine learning techniques.

### FIELD

This invention relates to a method and system for calculating physiological parameters from human body using multiple communication device sensors simultaneously.

### BACKGROUND

The demand for portable and reliable healthcare solutions has seen significant growth in recent years, driven by technological advancements in mobile devices and a societal shift towards personal health management. Traditional methods of physiological measurement often involve specialized equipment and healthcare settings, which can be inconvenient, time-consuming, and costly for users.

A smartphone is a communication device equipped with advanced computing capabilities. In recent years, smartphones have revolutionized the lives of humanity by evolving from mere communication device to more powerful tools capable of supporting a wide range of healthcare applications. Smartphones present a promising platform for physiological measurement and monitoring. These devices are now capable of capturing a variety of physiological signals using built-in sensors, such as cameras and microphones, and additional sensors like gyroscopes, barometers, ambient light sensors etc. Consequently, the ability to perform physiological assessments using everyday devices like smartphones and mixed reality devices offers an attractive alternative for both users and healthcare providers.

Currently, most smartphone-based physiological measurements rely on a single signal source, typically involving a simple interaction with the device's camera or microphone. Single-source measurements can be prone to errors caused by noise, movement artifacts, or changes in ambient conditions. The reliance on the single signal source may give rise to reliability and accuracy issues and may necessitate repeated measurements for ensuring better accuracy leading to user inconvenience and decreased trust in the smart phone-based health applications.

Further, the existing smartphone based physiological measurement applications focus on a narrow range of physiological parameters, such as heart rate or blood oxygen levels, without the ability to comprehensively assess multiple health indicators simultaneously, thereby leading to restricted capability. Additionally, the existing smartphone based physiological measurement applications require specific user positioning, thereby limiting the convenience of the user.

Therefore, there is a need for a unique solution that addresses the problem of the existing smartphone based physiological measurement such as low reliability and accuracy and user inconvenience. These challenges underscore the need for a more robust solution that leverages the full potential of smartphone technology to deliver reliable, multi-faceted physiological measurements.

### SUMMARY

The above-mentioned shortcomings, disadvantages and problems are addressed herein, which will be understood by reading and studying the following specification.

### BRIEF DESCRIPTION OF THE FIGURES

Embodiments herein are illustrated in the accompanying drawings, throughout which like reference letters indicate corresponding parts in the various figures. The example embodiments herein will be better understood from the following description with reference to the drawings, in which:

FIG. 1 illustrates a block diagram showing different components of a system for calculating physiological parameters from human body using multiple communication device sensors simultaneously, in accordance with an embodiment of the present invention.

FIGS. 2A-2C illustrate an implementation of hybrid remote-plethysmography, utilizing one or more fingertips for contact-based measurements in conjunction with remote video analysis to capture physiological parameters, in accordance with an embodiment of the present invention.

FIG. 3 illustrates a flowchart showing a method of calculating physiological parameters from human body using multiple communication device sensors simultaneously, in accordance with an embodiment of the present invention.

### DETAILED DESCRIPTION

The embodiments herein and the various features and advantageous details thereof are explained more fully with reference to the non-limiting embodiments and detailed in the following description. Descriptions of well-known components and processing techniques are omitted so as not to unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Some embodiments of this disclosure, illustrating all its features, will now be discussed in detail. The words “enabling”, “establishing”, and other forms thereof, are intended to be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise. The terms “comprises,” “comprising,” “has,” “having,” “includes” and/or “including” as used herein, specify the presence of stated features, elements, and/or components and the like, but do not preclude the presence or addition of one or more other features, elements, components and/or combinations thereof. The term “an embodiment” is to be read as “at least one embodiment.” The term “another embodiment” is to be read as “at least one other embodiment.” Although any system and methods similar or equivalent to those described herein can be used in the practice or testing of embodiments of the present disclosure, the exemplary system and methods are now described.

The disclosed embodiments are merely examples of the disclosure, which may be embodied in various forms. Various modifications to the embodiment will be readily apparent to those skilled in the art and the generic principles herein may be applied to other embodiments. However, one of ordinary skill in the art will readily recognize that the present disclosure is not intended to be limited to the embodiments described but is to be accorded the widest scope consistent with the principles and features described herein.

The detailed description set forth below in connection with the appended drawings is intended as a description of various implementations of the present disclosure and is not intended to represent the only implementations in which details of the present disclosure may be applied. Each implementation described in this disclosure is provided merely as an example or illustration, and should not necessarily be construed as preferred or advantageous over other implementations.

There is a need for a system that addresses the problem of low reliability, accuracy and user inconvenience in measurement of physiological parameters from human body.

It must be understood that reference of any specific application in current disclosure, such as the physiological parameter measurement application, is merely provided for the ease of explanation, and should not be construed as a limiting factor for application of the methodologies described herein. Therefore, it is fairly possible for a person skilled in the art to utilize the details provided in current disclosure for any similar application.

Communication device as described in the present invention may include but not limited to a mobile phone and mixed-reality headsets.

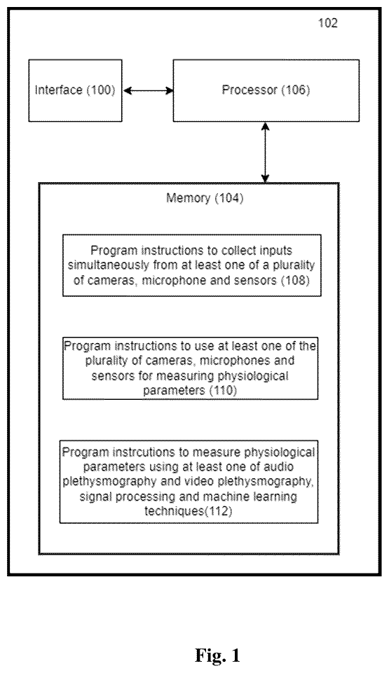

FIG. 1 illustrates a block diagram showing different components of a system 102 for measuring physiological parameters from human body using multiple communication device sensors simultaneously, in accordance with an implementation of the present invention. The system 102 includes the memory 104, the processor 106, and an interface 100. The memory 104 may store program instructions to perform several functions for calculating physiological parameters from human body using multiple communication device sensors simultaneously. For example, program instructions stored in the memory 104 may include program instructions to collect inputs simultaneously from at least one of a plurality of cameras, microphones and sensors 108, program instructions to use at least one of the plurality of cameras, microphones and sensors for measuring physiological parameters 110, program instructions to measure physiological parameters using at least one of audio plethysmography and/or video plethysmography, signal processing and machine learning techniques 112. The processing of the sensor data may be carried out on the communication device and cloud.

The program instructions to use at least one of the plurality of cameras, microphones and sensors of the communication device for measuring physiological parameters 110 may be executed by employing at least one of the plurality of cameras, microphones and sensors of the communication device. The measurement may be done by placing one or more fingertips on rear and/or front cameras and microphone or using the front camera to capture facial video data and along with that optionally capture audio data from the fingertips.

The program instruction to capture sensor data may cause the processor 106 to analyze the captured signals using machine learning algorithms to provide real-time feedback on the physiological parameters. Further, the program instruction to capture sensor data may cause the processor 106 to employ signal processing techniques to filter noise from captured audio and video signals to improve quality of data of the physiological parameters. The communication device may include one or more sensors to enhance accuracy of measurement of the physiological parameters. Further, the measured physiological parameters may be stored and transmitted to remote health monitoring systems. Additionally, the measured physiological data may be encrypted for secure transmission and storage. Further, the system may be integrated with third-party health applications for comprehensive health monitoring and management.

In one embodiment, the processor further analyses the captured signals using machine learning algorithms to provide real-time feedback on the physiological parameters.

In yet another embodiment, the processor employs signal processing techniques to filter noise from captured audio and video signals to improve quality of data of the physiological parameters.

In yet another embodiment, the communication device includes at least one of sensors of same type or different types to enhance accuracy of measurement of the physiological parameters.

In yet another embodiment, the processor is further configured to store and transmit at least one of raw data, raw signal and measured physiological parameters to remote health monitoring systems.

In yet another embodiment, measured physiological data is encrypted for secure transmission and storage.

In yet another embodiment, the processor is further configured to integrate with third-party health applications for comprehensive health monitoring and management.

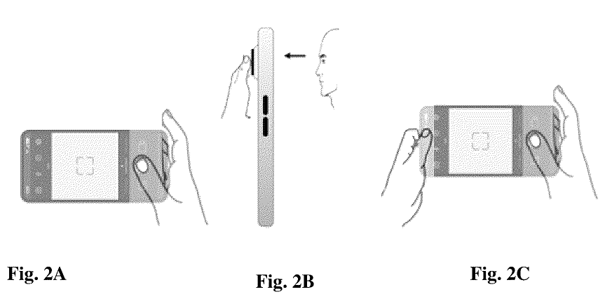

FIG. 2 illustrates an implementation of hybrid remote-plethysmography, utilizing one or more fingertips for contact-based measurements in conjunction with remote video analysis to capture physiological parameters, in accordance with an embodiment of the present invention. In this embodiment, fingertips may be placed on the rear cameras while the front camera records the face as illustrated in FIG. 2a and FIG. 2b. Fingertips from the opposite hand can simultaneously capture audio signals if microphones are positioned at the other end of the smartphone as illustrated in FIG. 2c. This hybrid approach utilizes both direct-contact sensors and non-contact video techniques, ensuring greater accuracy and improving the reliability of physiological measurements. Similar implementation may also be used for multi-site photoplethysmography.

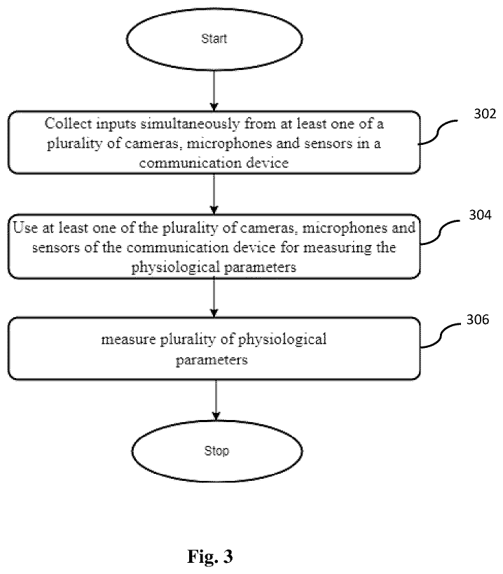

FIG. 3 illustrates a flowchart showing a method of measuring physiological parameters from human body using multiple communication device sensors simultaneously, in accordance with an implementation of the present invention. At step 302 inputs are collected simultaneously from at least one of a plurality of cameras, microphones and sensors of the communication device for enhancing the accuracy of the measurement of the physiological parameters. At step 304, at least one of the plurality of cameras, microphones and sensors of the communication device are used for measuring physiological parameters by placing one or more fingertips on rear and/or front cameras and microphone or using the front camera to capture facial video data and capture audio data from the fingertips. At step 306, the physiological parameters are measured using at least one of audio plethysmography and/or video plethysmography, signal processing and machine learning techniques.

In one embodiment, the captured signals are analyzed using at least one of signal processing techniques and machine learning algorithms to provide real-time feedback on the physiological parameters.

In another embodiment, at least one of signal processing techniques and machine learning techniques are employed to filter noise from captured audio and video signals to improve quality of data of the physiological parameters.

In another embodiment, one or more sensors of same type or different types are used to enhance accuracy of measurement of the physiological parameters.

In yet another embodiment, the measured physiological parameters are stored and transmitted to remote health monitoring systems.

In another embodiment, the measured physiological data is encrypted for secure transmission and storage.

In yet another embodiment, integration with third-party health applications is provided for comprehensive health monitoring and management.

The integration of direct-contact sensors with non-contact video methods in the present invention ensures higher accuracy, thereby enhancing the reliability of physiological measurements.

The method is illustrated in FIG. 3 as a collection of operations in a logical flow graph representing a sequence of operations that can be implemented in hardware, software, firmware or a combination thereof.

The term “software” as used herein is intended to encompass such instructions stored in storage medium such as RAM, a hard disk, optical disk, cloud hosted or so forth, and is also intended to encompass so-called “firmware” that is software stored on a ROM or so forth. Such software may be organized in various ways, and may include software components organized as libraries, Internet-based programs stored on a remote server or so forth, source code, interpretive code, object code, directly executable code, and so forth. It is contemplated that the software may invoke system-level code or calls to other software residing on server or other location to perform certain functions.

An embodiment of the invention may be an article of manufacture in which a machine-readable medium (such as microelectronic memory) has stored thereon instructions which program one or more data processing components (generically referred to here as a “processor”) to perform the operations described above. In other embodiments, some of these operations might be performed by specific hardware components that contain hardwired logic (e.g., dedicated digital filter blocks and state machines). Those operations might alternatively be performed by any combination of programmed data processing components and fixed hardwired circuit components.

An advantage of the present invention is that a method and system is provided for measuring physiological parameters from human body using multiple communication device sensors simultaneously.

As used in the present specification, the term “artificial intelligence” refers broadly to an artificial intelligence technique in which a computer's behavior evolves based on empirical data. In some cases, input empirical data may come from databases and yield patterns or predictions thought to be features of the mechanism that generated the data. Further, a major focus of artificial intelligence is the design of algorithms that recognize complex patterns and makes intelligent decisions based on input data. Artificial Intelligence may incorporate a number of methods and techniques such as; supervised learning, unsupervised learning, reinforcement learning, multivariate analysis, case-based reasoning, backpropagation, and transduction.

A processor may include one or more general purpose processors (e.g., INTEL® or Advanced Micro Devices® (AMD) microprocessors) and/or one or more special purpose processors (e.g., digital signal processors or Xilinx® System On Chip (SOC) Field Programmable Gate Array (FPGA) processor), MIPS/ARM class processor, a microprocessor, a digital signal processor, an application specific integrated circuit, a microcontroller, a state machine, or any type of programmable logic array.

A memory may include but is not limited to, non-transitory machine-readable storage devices such as hard drives, magnetic tape, floppy diskettes, optical disks, Compact Disc Read-Only Memories (CD-ROMs), and magnetooptical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions.

Any combination of the above features and functionalities may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set as claimed in claims that issue from this application, in the specific form in which such claims issue, including any subsequent correction.

In the above description, for purposes of explanation, numerous specific details are set forth in order to provide a thorough understanding of the present systems and methods. It will be apparent the systems and methods may be practiced without these specific details. Reference in the specification to “an example” or similar language means that a particular feature, structure, or characteristic described in connection with that example is included as described, but may not be included in other examples.

The foregoing description of the specific embodiments will so fully reveal the general nature of the embodiments herein that others can, by applying current knowledge, readily configure and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

I claim:

1. A system for measuring physiological parameters of a human body using hybrid remote audio video plethysmography techniques, comprising:
one or more processors; and
one or more memories coupled with the one or more processors, the one or more memories storing programmed instructions, which when executed by the one or more processors, causes the one or more processors to:
collect inputs simultaneously from at least one of a plurality of cameras, microphones and sensors in a communication device for enhancing the accuracy of the measurement of the physiological parameters;
use at least one of the plurality of cameras, microphones and sensors of the communication device for measuring the physiological parameters by placing one or more fingertips on rear and/or front cameras and microphone or using the front camera to capture facial video data and capture audio data from the fingertips; and
measure plurality of physiological parameters simultaneously or asynchronously using at least one of audio plethysmography and/or video plethysmography, signal processing and machine learning techniques.

2. The system of claim 1, wherein the processor is further configured to analyze the captured signals using machine learning algorithms to provide real-time feedback on the physiological parameters.

3. The system of claim 1, wherein the processor employs at least one of signal processing and machine learning techniques to filter noise from captured audio and video signals to improve quality of data of the physiological parameters.

4. The system of claim 1, wherein the communication device includes the plurality of sensors to enhance accuracy and reliability of measurement of the physiological parameters.

5. The system of claim 1, wherein the processor is further configured to store and transmit at least one of raw data, raw signal and measured physiological parameters to remote health monitoring systems.

6. The system of claim 1, wherein measured physiological data is encrypted for secure transmission and storage.

7. The system of claim 1, wherein the processor is further configured to integrate with third-party health applications for comprehensive health monitoring and management.

8. A method for measuring physiological parameters of a human body using hybrid remote audio video plethysmography techniques, comprising:
collecting inputs simultaneously from at least one of a plurality of cameras, microphones and sensors in a communication device for enhancing the reliability and accuracy of the measurement of the physiological parameters; using at least one of the plurality of cameras, microphones and sensors of the communication device for measuring the physiological parameters by placing one or more fingertips on rear and/or front cameras and microphone or using the front camera to capture facial video data and capture audio data from the fingertips; and
measuring a plurality of physiological parameters simultaneously or asynchronously using a combination of at least one of audio plethysmography and/or video plethysmography, signal processing and machine learning techniques.

9. The method of claim 8, wherein the processor is further configured to analyze the captured signals using at least one of signal processing techniques and machine learning algorithms to provide real-time feedback on the physiological parameters.

10. The method of claim 8, wherein the processor employs at least one of signal processing techniques and machine learning techniques to filter noise from captured audio and video signals to improve quality of data of the physiological parameters.

11. The method of claim 8, wherein the communication device includes at least one of sensors of same type or different types to enhance reliability and accuracy of measurement of the physiological parameters.

12. The method of claim 8, wherein the processor is further configured to store and transmit measured physiological parameters to remote health monitoring systems.

13. The method of claim 8, wherein measured physiological data is encrypted for secure transmission and storage.

14. The method of claim 8, wherein the processor is further configured to integrate with third-party health applications for comprehensive health monitoring and management.
